(19) United States
(12) Patent Application Publication
(10) Pub. No.: US 2009/0304808 A1
(43) Pub. Date: Dec. 10, 2009

(54) Anti-cancer immune-modulating agent

(75) Inventors: Armen Kazanchian, Glendale, CA (US); Robert Khachatryan, Ijmuiden (NL)

Correspondence Address: WESTMAN CHAMPLIN & KELLY, P.A., SUITE 1400, 900 SECOND AVENUE SOUTH, MINNEAPOLIS, MN, 55402, US

(73) Assignee: Montco Corporation, Curacao (NL)

(21) Appl. No.: 12/173,385

(22) Filed: Jul. 15, 2008

(30) Foreign Application Priority Data
Jun. 5, 2008 (EP) PCT/EP2008/057036

(51) Int. Cl. A61K 35/48; A61P 31/00

(52) U.S. Cl. 424/583

(57) ABSTRACT

The present invention discloses a method for the preparation of an anti-cancer composition from umbilical cord tissue and animal embryonic tissues, the latter preferably obtained from different animal classes. The composition obtainable by the method comprises different embryonic antigens and is used in therapy and prophylaxis of any type of cancer.

### CROSS-REFERENCE TO RELATED APPLICATION

The present application claims priority of patent application Serial No. PCT/EP2008/057036, filed Jun. 5, 2008, and published in English the content of which is hereby incorporated by reference in its entirety.

### FIELD OF THE INVENTION

The present invention relates to the field of treatment and/or prevention of cancer.

### BACKGROUND OF THE INVENTION

Currently, the problem of malignant diseases still is one of the most difficult ones humanity is facing. Despite the technological achievements in early recognition, extensive preventive approaches along with life style modifications, new advances in chemotherapy and other therapeutic modalities, medicine still cannot offer a definite way to fight cancer.

It is well known that anti-cancer medications may substantially differ from each other and represent a variety of antibiotics, alkyl compositions, hormonal preparations, enzymes, different substances of animal and non-animal origin, etc. The mechanism of action of each group of anti-cancer preparations is different. The majority acts at the level of DNA/RNA by disrupting its normal replication and proliferation. Others interfere with cellular metabolism. There is no “universal” available agent acting at multiple levels.

Recent interest to therapeutic agents from embryonic source was justified by better efficacy, less toxicity and good tolerability. The downside of these agents (currently in use) is high antigen composition of the agent, which can trigger severe allergic reaction. Although some non-scientific issues arose in relation to these agents (ethical, religious etc.) nevertheless they form the most promising group of therapeutic agents in giving the final solution to treat and prevent malignancies.

The immunogenicity of tumor related antigens is weak compared to microbial, viral and other antigens. In order to express fully their immunogenicity, the antigens should be associated with major histocompatibility complex 1 and 2 antigens. In this way, T-cells are being triggered to fight the cancer cells recognized as foreign ones.

Most anticancer agents do not provide universal therapeutic and/or preventive measures, probably because they are designed to fight specific types of cancer.

### SUMMARY OF THE INVENTION

The present invention aims at delivering universal protection against cancer development as well as to mobilize the body's resources to fight an existing malignant state. The anticancer composition disclosed in the present invention comprises particular embryonic antigens that also are found as cancer antigens in various tumor types. The composition surprisingly shows therapeutic activity against multiple cancer types. In addition, the composition of the invention can be used as a universal anti-cancer vaccine.

In a first aspect, the present invention provides a method for the preparation of a composition suitable for use as an anti-cancer agent (ACA). In the context of the invention, the term “anti-cancer” includes a cancer-preventive as well as a cancer-therapeutic activity of the composition. The composition is obtainable from a mixture of human umbilical cord tissue and animal embryonic tissues.

In particular, the method for the preparation of the composition comprises:
a) homogenizing animal embryonic tissue and human umbilical cord tissue at a temperature of ≦−50° C., to obtain a powder;
b) diluting the powder with water, to obtain a homogenate;
c) irradiating the homogenate with UV light;
d) precipitating protein from the irradiated homogenate by adding ethanol to the homogenate and recovering the precipitate;
e) dissolving the precipitate in water to obtain a protein solution;
f) optionally removing debris from the solution;
g) subjecting the solution to ultrafiltration to obtain an ultrafiltrate;
h) drying the ultrafiltrate to obtain the anti-cancer composition.

### DETAILED DESCRIPTION

Human umbilical cord tissue and any animal embryonic tissue may be used as starting material. In the context of the invention, the terms “animal embryonic tissue” and “animal embryos” refer to embryonic tissues as well as complete animal embryos, but exclude embryonic tissue or complete embryos from human origin.

Preferably, any animal species may be used as source of the embryonic tissue. Preferably, a mixture of embryos from different animal classes is used, for instance mammalian embryos, fish embryos and/or bird embryos. The use of embryos from different animal classes advantageously allows the provision of the appropriate levels of the different embryonic antigens in the composition according to the invention.

The tissues are preferably provided in a weight ratio of approximately 1 part of animal embryonic tissue and approximately 1-10 parts of human umbilical cord tissue, preferably approximately 1 part of animal embryonic tissue and approximately 2-5 parts of human umbilical cord tissue, more preferably approximately 1 part of animal embryonic tissue and approximately 3 parts of human umbilical cord tissue.

When animal embryos from different animal classes are used, they are preferably provided in the following weight ratios: approximately 1 part of bird embryos, approximately 1.5-2.5 parts of mammalian embryos, approximately 1.5-2.5 parts of fish embryos, and mixed with approximately 10-20 parts of human umbilical cord tissue. An especially preferred weight ratio is approximately 1 part of bird embryos, approximately 2 parts of mammalian embryos, approximately 2 parts of fish embryos, and approximately 15 parts of umbilical cord tissue.

In a preferred embodiment, the mammalian embryos are pig embryos, the bird embryos are chicken embryos and the fish embryos are trout embryos.

The embryos are preferably used when being at about a quarter of their gestational period. For instance, pig embryos may be at approximately 26-30 days of gestation and chicken embryos may be at approximately 6-8 days of gestation. Fish embryos may be at approximately 4-6 days of gestation.

Human umbilical cord tissue is washed in running water directly after birth of the baby to remove blood residues. It may be stored frozen for a certain time period, e.g. approximately 2-3 weeks, preferably up to approximately 1 week, prior to further processing.

The animal embryos may also be stored frozen prior to use.

The (thawed) umbilical cord tissue is immersed in 70% ethanol for at least 1 hour. Also the animal embryos used are being immersed in 70% ethanol.

The umbilical cord tissue and embryos are cut into pieces of ≦1 cm and mixed in the desired ratio, e.g. as indicated above.

The tissue pieces are frozen at a temperature ≦50°, preferably in liquid nitrogen, and then are homogenized to achieve a powder condition. Any suitable homogenizer may be used, preferably a homogenizer allowing homogenization with high pressure. To control the quality of disintegration of the tissue, the presence of fragmented cellular nuclear as well as cytoplasmatic debris is judged by microscopic evaluation. The cells should be disintegrated as much as possible.

The thus-obtained powder is diluted about 1:2 to about 1:5 with water, to obtain a homogenate.

The water used in the present invention preferably is chemically pure water, e.g. distilled water.

The homogenate is irradiated with UV irradiation. This is preferably done in a shallow bed with a depth of approximately 2 to 5 cm, more preferably with a depth approximately 2 to 3 cm. The wavelength of the UV light preferably is approximately 250 to 260 nm.

Any debris that is formed is removed from the UV-irradiated homogenate, for instance by centrifugation in a refrigerated centrifuge at approximately 15,000-20,000 g.

A protein fraction then is precipitated from the homogenate. This is preferably done by alcohol precipitation, more preferably using ethanol. To that end, the homogenate is mixed with approximately 2 to 3 weight parts of 96% ethanol and protein is allowed to precipitate. Typically, complete precipitation is reached in about half an hour. The alcoholic mixture is centrifuged at high speed, e.g. at least at 20,000 g, to recover the precipitate.

The obtained precipitate is suspended in water and incubated under agitation to dissolve (most of) the precipitate. Preferably, the precipitate is suspended in approximately 1 to 5 weight parts, more preferably in approximately 3 to 5 weight parts, of water. A suitable time period for incubation for instance is approximately 5 to 15 minutes. Any debris that may be present is removed, for instance by centrifugation at high speed (at least 20,000 g). The water preferably is distilled water or any other form of chemically pure water.

The resulting solution is subjected to ultrafiltration over a membrane with a molecular weight exclusion limit of 10-20 kDa, preferably of 10 kDa.

Optionally, prior to ultrafiltration the solution may be subjected to a prefiltration, for instance over a ceramic membrane with a pore size of approximately 0.5-1.5 mm.

The resulting ultrafiltrate is dried, preferably by lyophilization.

The present invention also envisages separate processing of animal embryonic tissues and umbilical cord tissue and,mixing in the appropriate ratios either at an intermediate stage of the process, for instance at the homogenate stage, or after dissolving the final dried product in water.

The composition obtainable by this method forms a second aspect of this invention.

The present invention provides a composition that advantageously contains a combination of embryonic antigens. In particular, the present invention discloses a composition comprising cancer embryonic antigen (CEA), alpha-fetoprotein (Alpha-FP), chorionic gonadotropin (CG), thyroxine-binding globulin (TBG), cancer antigen 125 (CA-125), and cancer antigen 19 (CA-19), respectively, in a ratio of 0.5-5 ng, 30-300 ng, 0.0005-0.015 IU, 15-150 ng, 5-45 U, and 30-300 U, respectively.

Preferably, the composition comprises cancer embryonic antigen (CEA), alpha-fetoprotein (Alpha-FP), chorionic gonadotropin (CG), thyroxine-binding globulin (TBG), cancer antigen 125 (CA-125), and cancer antigen 19 (CA-19), respectively, in a ratio of 1-2 ng, 90-110 ng, 0.001-0.01 IU, 40-60 ng, 10-20 U, and 70-1 10 U, respectively.

The above ratio is preferably measured on a solution of 5 mg of the dry composition diluted in 2 ml of distilled water.

The composition is suitable for use in therapy. In particular, such use relates to the prevention and/or treatment of all kinds of malignancies.

The composition of the invention comprises about 40-70% (w/w) protein, based on dry weight of the composition. The remainder is mainly carbohydrate. Most of the carbohydrate is conjugated to protein, and about 10-20% of the carbohydrate is free carbohydrate.

The dry composition is a sterile, white or light-yellow, cotton-like substance.

The composition of the invention may be administered subcutaneously, intravenously or topically.

For an injectable product, the composition of the invention is preferably mixed into normal saline (sterile 0.9% NaCl), to form an opaque, homogeneous fluid with a slightly yellow color, preferably having a concentration of 0.005-0.1% (w/w). Preferably, the injectable product is administered, optionally after dilution with normal saline, via subcutaneous injection in doses of 0.01-0.05 mg/kg. Preferred injection site is an inner surface of the forearm. The skin reaction at the site of injection reveals the ability of the body's immune system to respond to the composition.

Patients with established malignancies may be treated once every 3-6 months with the same dosing regime. It is recommended to give additional booster doses during the first month of the therapy in severe cases and/or to give extra doses in patients with advanced malignant states.

For prophylaxis, the composition of the invention typically is administered to an individual with risk factors and high predisposition to oncological diseases (family history, smoking, exposure to carcinogenic substances, history of previous cancer, high fibrinogenic states etc.) by one injection per year, also using doses of 0.01-0.05 mg/kg.

The composition of the invention also may be topically applied as a cream or an ointment. The concentration of ACA in the cream or ointment may be 0.1-0.5% It was surprisingly found that the composition has profound juvenile effect when applied to the skin.

The composition (ACA) is effectively used for the treatment of different kinds of human cancers including solid and non-solid tumors, leukemia, etc. and achieves steady therapeutic effect including full recovery from the cancerous state.

The composition (ACA) is also successfully used to prevent development of cancer in patients with heavy predisposition and risk factors to develop a cancerous state.

The composition of the invention is especially suitable to treat and/or to prevent neoplastic and pre-neoplastic lesions of the skin and mucous membranes.

The composition of the invention never triggered major side effects or complications upon administration.

### EXAMPLE 1

### Preparation of ACA

Umbilical cord tissue after cleaning in running water (4 hours) was immersed in approximately 70% ethanol for approximately 1.5 hours. Then it was mixed with pig embryos, trout embryos and chicken embryos, all immersed in alcohol, in following % of weight: approximately 75% human umbilical cord, approximately 10% pig embryos, 10% fish embryos and approximately 5% chicken embryos. The embryos of pig were at approximately 26-30 days of gestation, embryos of trout were at approximately 4-6 days of gestation and embryos of chicken were at approximately 6-8 days of gestation. The tissues were cut into pieces ≦1 cm, mixed and frozen in liquid nitrogen.

The frozen pieces then were homogenized in a homogenizer with a high pressure force to achieve a powder condition.

The powder was then suspended in distilled water 1:4 and centrifuged with speed up to 400 rpm in a ball rotary centrifuge, where the contact surfaces were made from inert material. The quality of disintegration of the tissue was controlled by checking the presence of fragmented cellular nuclear as well as cytoplasmic debris, by microscopic evaluation.

The homogenate was subjected to UV irradiation in a shallow bed of about 3 cm for approximately 30 minutes, after which it was again centrifuged in a refrigerated centrifuge with speed of 6000 rpm for a period of 15 minutes.

Proteins, among which glycoproteins, were precipitated from the supernatant by adding 2-3 weight parts 96% ethanol and allowing the mixture to stand for at least half an hour. The mixture was then centrifuged at high speed up to 20,000 rpm for 20 minutes.

The precipitate was suspended 1:2 in distilled water and, after 10 minutes exposure in ball rotary centrifuge at 400 rpm and 37° C., was again centrifuged at a high speed (20,000 rpm) in zonal rotary centrifuge CR-65.

The supernatant was subjected to a macrofiltration step using a ceramic membrane with a pore size of 1.4 mm (Jiangsu Jiuwu Hi-tech Co. Ltd, China)). Then, the filtrate was subjected to ultrafiltration using a modified polyethersulfone membrane with a molecular weight exclusion limit of 10 kDa (AC080R01, AMC, USA). The resulting ultrafiltrate was subjected to drying by lyophilization.

Freezing was done in a rotatory sterile chamber at −25 to −30° C. for 2 hours. The preparation then was exposed to −38 to −40° C. in a freezing chamber for 24 hours.

The final product was obtained by gradual increase of the temperature from −40 to room temperature over 28-30 hours.

### EXAMPLE 2

### Composition and Characteristics of ACA

In the lyophilized product, the concentration of protein constitute about 60% of the weight of ACA (by spectrophotometer analysis). About 1.5% constitutes free carbohydrates, where 8-10% is conjugated carbohydrates.

There are some enzymes and minerals present in the product, measured when dissolving 5 mg dry product in 2 ml distilled water:

| | | |
|---|---|---|
| Alanine amino transferase (ALT) | 1.4 | U/l |
| Aspartate amino transferase (AST) | 71.9 | mmol/l |
| Lactate dehydrogenase (LDH) | 3.0 | U/l |
| Alkaline phosphatase (APT) | 24.7 | U/l |
| Mg | 1.7 | mmol/l, |
| K | 0.96 | mmol/l, |
| Ca | 0.65 | mmol/l, |
| P | 0.52 | mmol/l. |

The monoclonal immunoassay revealed presence of the following cancer embryonic compounds (the sample size was 5 mg of the composition dissolved in 2 ml of distilled water):

| | | |
|---|---|---|
| Cancer embryonic antigen (CEA) | 1.4-1.7 | ng/ml |
| Alpha-fetoprotein (Alpha-FP) | 95-99 | ng/ml |
| Chorionic gonadotropin (CG) | 0.0022-0.0055 | IU/ml |
| Thyroxine-binding globulin (TBG) | 45-50 | ng/ml |
| Cancer antigen 125 (CA-125) | 13.5-14.3 | U/ml |
| Cancer antigen 19 (CA-19) | 79.8-102.2 | U/ml |

CG, TBG and AFP were detected by CMIA (chemi-luminescent immuno-assay on Abbot Architect analyzer) and CEA, CA-19 and CA-125 were detected by MEIA (microparticle enzyme immuno-assay on Abbot AxSYM analyzer).

These embryonic antigens are similar to multiple cancer antigens and cross-react with them.

Along with embryonic antigens there are some other types of proteins in ACA which were detected by electrophoresis, chromatography, etc. These proteins most probably were responsible for some interferon-like, anti-virus and anti-mutagen characteristics of the product (ACA).

ACA is a porous, soft substance having a light yellow and/or white color without any smell or taste.

The injectable product is a 0.02%, opaque, homogeneous fluid with a slightly yellow color.

### EXAMPLE 3

### Toxicity, Mutagenesis

The toxicity experiments were performed on 24 dogs who were receiving doses of ACA of one human therapeutic dose, and of 10-fold and 25-fold of a human therapeutic dose.

The dogs were monitored 7, 15 and 30 days after injections. The baseline parameters were registered before the start of the experiment (EKG, temperature, RR, urine analyses, CBC, full chem.-21 including AST, ALT, APT, LDH etc.

Mutagenesis was tested on rats according to WHO recommendations. The ACA was introduced to the rats 5-15 days before application of benzpirena and or 7.12 dimethilbenzantracena. The study of rats bone marrow showed definite decrease of megakariocytes with chromosomal aberrations (p<0.001) in comparison with control group (only cancerogens).

### EXAMPLE 4

Treatment of Patients with ACA

Preventive approach: to the high risk group of the patients—one vial s/q once a year.

Treatment with ACA: one vial per each injection, four times per year. The second injection should be given a month after the first one, then every 6 months.

The site of first injection usually develops local reaction in the way of redness 3-6 cm with some elevation and induration, which disappears after 48 hours. The local reaction can give as some idea about anti-cancer immune status of the patient, based on the fact that it is basically a set of cancer antigens. So if there is an intense local reaction, definitely the ability of the immune system to distinguish and react to cancer is in good shape.

The data obtained with therapy and prophylaxis are shown in Tables 1 and 2, respectively.

TABLE 1
Comparative data on effect of ACA for treatment
of different cancer states

| ACA treatment group | | | Conventional | |
|---|---|---|---|---|
| (5 years follow-up) | | | treatment group | |
| | | % of patients | | % of patients |
| Disease | Pts | improved | Pts | improved |
| Breast CA | 120 | 76% | 115 | 32% |
| (no surgery) | | | | |
| Stomach CA | 180 | I-II stage 88% | 160 | 42% |
| | | III-IV stage - 76% | | |
| Leukemia | 118 | 91% | 88 | 56% |
| (CLL) | | | | |
| Lung CA | 192 | 71% | 145 | 45% |
| Prostate CA | 128 | 82% | 114 | 52% |
| Colon CA | 136 | I-II stage - 91% | 119 | 56% |
| | | III-IV stage - 78% | | |
| Total | 874 | | 741 | |

TABLE 2
Comparative data on ACA as a vaccine in prevention of cancer states
in first line relatives of patients with diagnosed malignancies
The participants were followed for 7 years, or until being
diagnosed with cancer.

| | | Incidents | | Incidents of |
|---|---|---|---|---|
| Type of CA in | Participants | of CA in | Participants | CA in non- |
| first line | 1 s/q injection | prevention | without | prevention |
| relatives | per year | group | vaccination | group |
| Stomach, | 1132 | 2-5% | 907 | 17-23% |
| Pancreatic CA, | | | | |
| Colon CA | | | | |
| Leukemia/CLL | 366 | 6% | 298 | 19% |
| Breast CA | 476 | 5% | 421 | 21% |
| Prostate CA | 397 | 3% | 323 | 15% |
| Total | 2371 | | 1949 | |

Although the present invention has been described with reference to preferred embodiments, workers skilled in the art will recognize that changes may be made in form and detail without departing from the spirit and scope of the invention.

What is claimed is:

1. A method for the preparation of a composition suitable as an anti-cancer agent comprising:
a) homogenizing animal embryonic tissue excluding embryonic tissue or complete embryos from human origin and human umbilical cord tissue at a temperature of ≦−50° C., to obtain a powder;
b) diluting the powder with water, to obtain a homogenate;
c) irradiating the homogenate with UV light;
d) precipitating protein from the irradiated homogenate by adding ethanol to the homogenate and recovering the precipitate;
e) dissolving the precipitate in water to obtain a protein solution;
f) optionally removing debris from the solution;
g) subjecting the solution to ultrafiltration to obtain an ultrafiltrate;
h) drying the ultrafiltrate to obtain the anti-cancer composition.

2. The method according to claim 1, wherein the tissue is provided in a weight ratio of approximately 1 part of animal embryonic tissue and approximately 1-10 parts of human umbilical cord tissue.

3. The method according to claim 2 wherein the weight ratio comprises approximately 1 part of animal embryonic tissue and approximately 2-5 parts of human umbilical cord tissue.

4. The method according to claim 3 wherein the weight ratio ratio comprises approximately 1 part of animal embryonic tissue and approximately 3 parts of human umbilical cord tissue.

5. The method according to claim 1, wherein the animal embryonic tissue comprises mammalian, fish and/or bird embryonic tissue.

6. The method according to claim 5, wherein the mammalian embryonic tissue comprises pig embryonic tissue, the bird embryonic tissue comprises chicken embryonic tissue, and the fish embryonic tissue comprises trout embryonic tissue.

7. The method according to claim 5, wherein the tissues are provided in a weight ratio of approximately 1 part of bird embryos, approximately 1.5-2.5 parts of mammalian embryos, approximately 1.5-2.5 parts of fish embryos and approximately 10-20 parts of human umbilical cord tissue.

8. The method according to claim 7 wherein the weight ratio comprises approximately 1 part of bird embryos, approximately 2 parts of mammalian embryos, approximately 2 parts of fish embryos, and approximately 15 parts of umbilical cord tissue.

9. The method according to claim 5, wherein the mammalian, bird and fish embryonic tissues are at approximately a quarter of their gestational period.

10. The method according to claim 6, wherein the pig embryonic tissue is at approximately 26-30 days of gestation, the trout embryonic tissue is at approximately 4-6 days of gestation and the chicken embryonic tissue is at approximately 6-8 days of gestation.

11. The method according to claim 1, wherein the precipitation of protein comprises mixing approximately 1 weight part of the homogenate with approximately 2 to 3 weight parts of 96% ethanol.

12. The method according to claim 1, wherein ultrafiltration occurs over a membrane with a molecular weight exclusion limit of 10-20 kDa.

13. A composition produced by the method of claim 1 characterized by its suppression of more than one cancerous condition.

14. A composition useful in therapy or prophylaxis of cancer, the composition comprising a mixture of human umbilical cord tissue and animal embryonic tissue processed and purified to obtain an anti-cancer component for use in therapy or prophylaxis of cancer.

15. The composition of claim 14 wherein the animal embryonic tissue comprises embryonic tissue from a mammalian source, a fish embryo, and/or bird embryos.

16. The composition of claim 14 wherein the embryonic tissue comprises a weight ratio of approximately one part of animal embryonic tissue, and approximately 1 to 10 parts of human umbilical cord tissue.

17. The composition of claim 16 wherein the human umbilical cord tissue comprises a range of approximately 2 to 5 parts.

18. The composition of claim 17 wherein the human umbilical cord tissue comprises approximately 3 parts.

19. The composition of claim 14 wherein the animal embryonic tissue comprises pig, trout and/or chicken embryos.

20. A composition comprising cancer embryonic antigen (CEA), alpha-fetoprotein (Alpha-FP), chorionic gonadotropin (CG), thyroxine-binding globulin (TBG), cancer antigen 125 (CA-125), and cancer antigen 19 (CA-19), respectively, in a ratio of 0.5-5 ng, 30-300 ng, 0.0005-0.015 IU, 15-150 ng, 5-45 U, and 30-300 U, respectively.

21. The composition of claim 20, which comprises an aqueous composition further comprising water up to 1 ml.
